(19) United States
(12) Patent Application Publication
(10) Pub. No.: US 2010/0029584 A1
(43) Pub. Date: Feb. 4, 2010

(54) PROCESS FOR PRODUCING A CELLULOSE-BASED FILM TO BE USED FOR SKIN AND TISSUE LESIONS

(76) Inventor: Gianfranco Merizzi, Torino (IT)

Correspondence Address: Steinfl & Bruno, 301 N Lake Ave Ste 810, Pasadena, CA, 91101, US

(21) Appl. No.: 12/520,848

(22) PCT Filed: Aug. 3, 2007
(86) PCT No.: PCT/IB2007/053063

§ 371 Date: Jun. 22, 2009

(30) Foreign Application Priority Data
Dec. 29, 2006 (IT) TO2006A000932

(51) Int. Cl. A61K 31/717; C12P 19/04; A61P 17/02

(52) U.S. Cl. 514/57; 435/101; 536/56

(57) ABSTRACT

The process for producing a cellulose film, to be used for covering, regenerating, repairing and healing of skin and tissue lesions, such as sores, ulcers, burns, wounds etc. by means of cultivation, in a culture medium comprising nitrogen and carbon sources, of microorganisms comprising the Acetobacter xylinum species; the culture medium moreover comprises at least one microorganism belonging to the Leuconostoc genus.

The present invention refers to a biotechnology process for producing a cellulose-based film, to be used for covering and regenerating skin and tissue lesions, and to a biosynthetic film thus obtained.

In particular, the invention refers to a process in which the aforesaid cellulose-based film is obtained by means of Acetobacter xylinum bacteria.

EP-A-0 114 481 describes the production of liquid-impregnated pads, useful as medication for wounds and burns, prepared from cellulose film produced microbially by means of Acetobacter xylinum culture. The process provides for the cultivation of the microorganism in static conditions (non-stirred culture); the bacteria are thus cultivated on the surface of the nutrient medium to form a consistent film, which generally has a 0.1 mm to approximately 15 mm thickness; the film thus obtained is removed from the nutrient medium, treated with sodium hydroxide to remove the bacteria, neutralised and washed with water to give a water-impregnated film of microbial cellulose.

WO86/02095 also describes the preparation of cellulose films, obtained from the cultivation of Acetobacter xylinum, used as artificial skin graft; a Acetobacter xylinum culture is inoculated in a culture medium containing nitrogen and carbohydrates as nutrients, medium in the specific examples being composed of an infusion of Tea Sinensis with added sugar; the process comprises the incubation of the microorganism at 28° C. for a period of about 36 hours, after which the film is removed from the culture medium and dehydrated at room temperature on supports, in distended state. This patent underlies a medication commercialised with the BIOFILL mark, useful in the treatment of various skin lesions.

U.S. Pat. No. 5,846,213 describes the preparation of cellulose-based medications having flexibility and mechanical properties similar to those of human skin. A cellulose film is prepared in advance by means of cultivation of Acetobacter xylinum and it is then dissolved in a solvent system comprising dimethylacetamide and a lithium salt so to obtain a solution from which a cellulose membrane is obtained by means of casting and coagulation in a gelling bath.

The object of the present invention is that of providing a bacteria-produced cellulose film, and a process for its production, which is usable as medication for skin lesions and is effective in creating a favourable environment for the care of the lesion.

Another object is to provide a cellulose film which has suitable mechanical characteristics, in particular high flexibility, similar to the mechanical properties of the human skin.

Forming the object of the invention, therefore, are a process and a crystalline cellulose-based film having the characteristics defined in the following claims.

In particular, the process according to the invention is characterised in that the biosynthetic cellulose-based film is obtained by means of cultivation, in a culture medium containing nitrogen and carbon sources, of microorganisms belonging to the Acetobacter xylinum species, in the presence of at least one microorganism belonging to the Leuconostoc genus.

In a preferred embodiment, the process is conducted by inoculating a yeast in the culture medium as well, yeast preferably chosen from among Saccharomyces cerevisiae, Schizosaccharomyces pombae and/or Saccharomyces malidevorans and mixtures of two or three of these.

Acetobacter is a gram-negative, rod-shaped bacterium, strictly aerobic. It is characterised for the capacity to produce multiple chains of poly-beta-1,4-glucan, chemically identical to cellulose. The cellulose chains in microfibril form are synthesised on the bacterial surface at sites outside the cell membrane. In the scope of the invention, any strain of Acetobacter xylinum is used which is capable of producing cellulose microfibrils, both in static culture and in stirred culture conditions, including the specific strains described in EP 0 228 779.

The genus Leuconostoc comprises bacteria of spherical form, distinguishable from the genus Lactococcus by the production of gas. All of the species of the genus Leuconostoc have a heterofermentative metabolism; the bacteria are widely diffused in nature, especially on substrates of vegetal origin. They have optimal growth temperatures in the range of 20° C. and 30° C. and are less acidophilic than the lactobacilli, preferring weakly acidic or neutral substrates, except regarding Leuconostoc oenos (recently reclassified as Oenoscoccus oenos), which is adapted to the low pH of wine and which is known as malolactic fermentation agent of wines.

Based on the tests carried out by the applicant, it is deemed that the presence of bacteria of the genus Leuconostoc, in the Acetobacter xylinum culture, considerably influences the production and growth of the biomass.

Film obtained by means of the process according to the invention is essentially composed of crystalline cellulose and is characterised by a relatively low average degree of polymerisation, generally in the range of 250 and 400. An important aspect of such film which characterises it with respect to classic crystalline celluloses is its insolubility in concentrated sulphuric acid. This aspect suggests that there are bonds between the single cellulose chains which make the product insoluble in concentrated sulphuric acid and difficult to hydrolyse.

In the process according to the invention, the cultivation of the abovementioned microorganisms is generally conducted at a temperature in the range of 20° C. and 36° C. and at a pH in the range of 1 and 6, preferably at a temperature from 26° C. to 30° C. and with pH in the range of 2 and 4.

The preferred culture medium for the symbiotic growth is constituted by cane sugar and/or by a liquid extract of cane sugar, as sources of carbon and nitrogen. The culture medium can moreover comprise sugar and acetic acid.

The culture is generally maintained in static conditions.

The film which forms is separated from the culture medium by means of per se known techniques and is dehydrated by drying.

When the film is rehydrated with physiological solution, it takes on characteristics of translucidity, flexibility and density similar to those of integral human skin; it is moreover equipped with selective permeability towards water vapour and gas and impermeability for water and bacteria. The film possesses an excellent adhesion capacity to the injured part; the occlusion which is generated protects the wound from the outside, preventing contamination, diminishing the pain and creating an ideal microenvironment for granulation and re-epithelialisation.

Such film is particularly recommended on burns, ulcers and sores of level I up to deep level II, without excess exudation; the film adheres perfectly to the injured part and is inert, non-toxic, non-allergenic and lacks latex.

### EXAMPLE 1

In a 500 L stainless steel container, the following are inserted: 250 kg of cane sugar extract, 20 kg of cane sugar and vinegar in a quantity such to bring the mixture pH to 3.5. There is then the inoculation of the culture medium at room temperature with Acetobacter xylinum and Leuconostoc microorganisms, particularly Leuconostoc oenos, in 30 ml overall quantity. During this process, the pH is brought to and maintained at 2.5 by means of the addition of cane sugar and vinegar. Upon completion, the biomass is washed and refined up to the obtainment of the liquid state by means of the use of an industrial pulveriser; the liquid thus obtained is poured on a cotton support and housed in stainless steel cases connected in series with each other and then to a vacuum system which, by drying the liquid part, determines the formation of a smooth and uniform film on the cotton support.

The cotton support with adhered film is subsequently placed in the oven and once the drying process is complete, the film is removed from the support and sent to storage. Depending on the quantity of liquid biomass and on the size of the used case, films can be obtained of different size and thickness: from 750 ml of biomass in the liquid stated placed in a case of 23×30 cm size, for example, a film of 21×27 cm size arises with an approximately 0.05 mm thickness.

### EXAMPLE 2

The procedure of example 1 is repeated at the same abovementioned conditions, using as inoculant, in addition to the abovementioned bacteria, also Saccharomyces cerevisiae and Schizosaccharomyces pombae.

The film obtained according to example 2 was subjected to analysis to verify the structure and determine the cellulose content.

The following analyses were conducted.
i) Recording the FT-IR spectrum (ASI React IR 1000 instrumentation with diamond cell —analysis of the sample as is—resolution of 4 cm⁻¹).

The recorded IR absorption signals are:
3340 cm⁻¹ OH stretching
2984 cm⁻¹ CH aliphatic stretching
1460-1200 cm⁻¹ CH₂ deformation, CH and CH₂ wagging and OH deformation in plane
1100-1000 cm⁻¹ CH₂O and CoC stretching
897 and 663 cm⁻¹ bands typical of cellulose.

The FT-IR spectrum therefore corresponds to that of cellulose.
ii) Analysis of the total carbohydrates by means of the HPLC method equipped with refraction index detector.

The methods used were those which are normally used to determine the cellulose and hemicellulose content.

The cellulose content was determined according to an internal method of the Stazione Sperimentale Carta Cartoni e Pasta per Carta (SSCCPC; Paper, Cardboard and Pulp Experimental Station) of Milan with total hydrolysis of the polysaccharides in concentrated sulphuric acid, followed by a measurement of the total reducing sugars (Somogyi-Nelson method) and determination of the glucose by enzymatic method.

The hemicellulose content was determined according to the SSCCPC internal method of selective hydrolysis of the hemicellulose in diluted sulphuric acid, followed by a measurement of the total reducing sugars (Somogyi-Nelson method).

The obtained results (expressed as percentage by weight with respect to the dry weight of the analysed sample) indicate that the obtained film is not soluble in the adopted conditions, conditions which usually lead to the hydrolysis of cellulose and hemicellulose.

The results obtained on the sample were the following:
1.7% of resinous substances extractible in cyclohexane/ethanol,
75% of residue insoluble after hydrolysis in concentrated (72%) sulphuric acid, followed by hydrolysis with diluted sulphuric acid (3%), or in experimental conditions which usually permit hydrolysing both the cellulose and hemicellulose into corresponding monosaccharides,
4.6% total reducing sugars determined on the solute of the hydrolysis,
1.8% of glucose (ascribable to cellulose),
2.8% total reducing sugars (ascribable to hemicellulose),
85% of residue insoluble after hydrolysis in 1 M sulphuric acid of the 75% of hydrolysis residue with 72% and 3% sulphuric acid (experimental conditions which usually permit selectively hydrolysing the hemicellulose to the corresponding monosaccharides),
2.5% total reducing sugars in the residue after hydrolysis in 1 M sulphuric acid (ascribable to hemicellulose).

In order to better define the real chemical structure of the film, an analysis was carried out through nuclear magnetic resonance at the solid state. The spectrum of the nucleus ¹³C was recorded by means of the DD CP-MAS (Dipolar Decoupling Cross-Polarization Magic Angle Spinning) technique.

This type of technique is necessary for reducing the dipolar couplings, and therefore for diminishing the signal bandwidth, a decoupling at high power and the rotation of the sample around an axis which is displaced with respect to the fixed axis by an angle of 54° 7′, called the Magic Angle. The latter expedient allows eliminating the dipolar and homonuclear interactions and the chemical shift anisotropy. In particular, the DD CP-MAS technique was employed to increase the intensity of the signals of the diluted nuclei and to eliminate the problems connected with the overly long relaxation times of these same nuclei.

In this experiment, the spin polarisation of the abundant nuclei is utilised, such as for example the protons, which is relatively strong so to increase the spin polarisation of diluted nuclei, such as for example ¹³C.

The measurement was carried out with the As ×300 Bruker instrument, equipped with a 4 mm probe, by making the sample rotate at 8 kHz speed.

As results from the NMR spectra, the spectral profile has the typical signals of the glycosidic repetitive unit of the cellulose, whose profile results analogous to that of a cellulose I obtained from Acetobacter.

Moreover, from the whole crystalline C₄ and amorphous C₄, it is obtained that the crystalline percentage is about 75%, thus a sufficient value to be judged a high crystallinity index; in a further series of tests, crystallinity percentages were determined in the range of 65% and 90%.

The average degree of polymerisation was measured with the viscosimetric method UNI 8282/94. The sample was completely soluble in the used solvent (cupriethylenediamine), with average degree of polymerisation of 290.

1. A process for producing a cellulose film, the process comprising
cultivating microorganisms comprising an Acetobacter xylinum species in a culture medium comprising nitrogen, carbon sources, and at least one microorganism of a Leuconostoc genus.

2. The process according to claim 1, wherein said culture medium further comprises one or more yeasts selected from the group consisting of Saccharomyces cerevisiae, Schizosaccharomyces pombae, and Saccharomyces malidevorans.

3. The process according to claim 1, wherein cultivation is carried out at a temperature in a range of between 20° C. and 36° C. and a pH in a range of between 1 and 6.

4. The process according to claim 1, wherein cultivation is carried out at a temperature ina range of between 26° C. and 30° C. and a pH in a range of between 2 and 4.

5. The process according to claim 1, wherein the culture medium comprises cane sugar and/or a liquid extract of cane sugar.

6. The process according to claim 1, wherein the culture medium comprises a pH corrector, the pH corrector being acetic acid.

7. The process according to claim 1, wherein the at least one microorganism of the Leuconostoc genus is Leuconostoc oenos.

8. A crystalline cellulose-based biological film, wherein said film comprises crystalline cellulose in microfibril form having an average degree of polymerisation in a range of between 250 and 400.

9. The crystalline cellulose-based biological film according to claim 8, wherein said film is insoluble in concentrated sulphuric acid.

10. The crystalline cellulose-based biological film according to claim 8, wherein said film has a degree of crystallinity in a range of between 65% and 90%.

11. A crystalline cellulose-based biological film obtainable by the process according to claim 1.

12. A cellulose based medication comprising the crystalline cellulose-based biological film of claim 8.

13. The cellulose based medication of claim 12, wherein the medication is suitable for covering, regenerating, repairing, or healing a skin or tissue lesion.

14. The cellulose based medication of claim 12, wherein said film is insoluble in concentrated sulphuric acid.

15. The cellulose based medication of claim 12, wherein said film has a degree of crystallinity in a range of between 65% and 90%.

16. A cellulose based medication comprising the crystalline cellulose-based biological film of claim 11.

17. A method of covering, regenerating, repairing, or healing a skin or tissue lesion, the method comprising applying the crystalline cellulose-based biological film of claim 8 to a skin or tissue lesion.

18. The method of claim 17, wherein said lesion is a burn, ulcer, sore, or any combination thereof.

19. A method of covering, regenerating, repairing, or healing a skin or tissue lesion, the method comprising applying the crystalline cellulose-based biological film of claim 11 to a skin or tissue lesion.

20. The method of claim 19, wherein said lesion is a burn, ulcer, sore, or any combination thereof.
